(19) United States
(12) Patent Application Publication
(10) Pub. No.: US 2023/0166259 A1
(43) Pub. Date: Jun. 1, 2023

(54) SAMPLE COLLECTION TRAY FOR MULTI-WELL PLATES

(71) Applicant: Roche Sequencing Solutions, Inc., Pleasanton, CA (US)

(72) Inventor: Jan BERKA, Pleasanton, CA (US)

(21) Appl. No.: 17/921,617

(22) PCT Filed: Apr. 29, 2021
(86) PCT No.: PCT/EP2021/061294

§ 371 Date: Oct. 26, 2022

Related U.S. Application Data
(60) Provisional application No. 63/018,938, filed on May 1, 2020.

(51) Int. Cl. B01L 3/00; B01L 9/00; C12M 1/32

(52) U.S. Cl. CPC B01L 3/5085; B01L 9/523; C12M 23/12; B01L 2300/0829; B01L 2200/025; B01L 2200/0621; B01L 2300/021; B01L 2300/047; B01L 2400/0409; B01L 2400/0457; B01L 2300/0851; B01L 2300/0858; B01L 2300/12

(57) ABSTRACT

The invention is a device for quantitatively collecting pooled samples from multiwell plates.

### FIELD OF THE INVENTION

The invention relates to the field of liquid handling and more specifically, liquid handling in multiwell plates popular in chemistry, biology and diagnostic procedures. The invention is a device and assembly for handling multiwell plates.

### BACKGROUND OF THE INVENTION

Many laboratory techniques involve manipulations of liquids or suspensions using standard disposable multiwell plates or microplates. Microplates exist in variety of versions, with many well configurations and shapes. Multiwell plates share a common footprint dimensions, allowing them to be used by laboratory liquid handlers (lab robots). The purpose of microplates is to allow for simultaneous reactions (e.g. PCR, incubations, cell culture growth etc.) to happen in parallel in multiple samples. The number of discrete wells ranges fro 2 to 1536, each well volume being as small as few microliters to about 10 ml. Some lab operations require collection of material from all plate wells. One approach is to simply invert the plate and discard the liquid, e.g., an unwanted supernatant. In some applications, the contents of the well are used in subsequent steps and need to be collected. The collection is accomplished typically by repeated pipetting contents of individual wells (either manually or robotically) into a container. Such process is slow, tedious and results in material losses during multiple transfers. In some applications, such as pooling sequencing libraries, uneven recoveries may bias the final composition of a pooled sample. In cell manipulations, uneven numbers of cells are recovered from each well, causing experimental variability. Until now, no suitable commercially available receptacles exist to collect and recover the material without losses.

Based on the foregoing, there is a need to improve the existing methods and apparatus for collecting contents of multiwell plates or microplates.

### SUMMARY OF THE INVENTION

The invention is a device for pooling and quantitatively collecting the contents of wells of a microwell plate with an optional ability to centrifuge.

In some embodiments, the invention is a sample collection device comprising: a standard multiwell base plate, and a tray placed on the side of the multiwell plate containing the well openings, the multiwell plate having a lip, a shoulder and a skirt, the tray having a lip, a shoulder, a body and a closed nozzle, wherein, the lip of the plate rests atop the lip of the tray and the shoulder of the plate is enclosed by the shoulder of the tray and the shoulder of the tray abuts the skirt of the plate. To support the plate on the tray, the lip of the plate and the lip of the tray each have a width and the width of the tray lip is not less than the width of the plate lip. In some embodiments, the tray further comprises legs extending from the body in the direction of the tray nozzle to enable the tray to be freestanding. The legs may comprise supporting flanges extending from the body of the tray. In other embodiments, the device further comprises an accessory frame, having the bottom and four side walls, wherein the lip of the tray rests atop the edge of the side wall and the thickness of the side wall is no less than the width of the tray lip. In some embodiments, the bottom of the accessory frame comprises an opening accommodating the nozzle of the tray. In other embodiments, the nozzle of the tray is enclosed within the accessory frame. In some embodiments, the tray is made from low adhesion polymer material. In some embodiments, the tray is made from moldable polymer material, for example, polycarbonates, polyesters, nylons, fluoropolymers, acrylic and methacrylic resins and copolymers, polysulphones, polyethersulphones, polyarylsulphones, polystyrenes, polyvinyl chlorides, chlorinated polyvinyl chlorides, polyolefins and copolymers thereof and polyurethanes. One example is low adhesion polypropylene. In some embodiments, the accessory frame is made from material selected from moldable polymer material, Teflon, polycarbonate or metal.

In some embodiments, the invention is a sample collection tray having a body with a rectangular top opening, and a closed nozzle, wherein the dimensions of the top opening match the dimensions of a top side of a standard multiwell plate with the wells of the plate facing into the tray. The tray may further comprise legs extending from the body in the direction of the nozzle and enabling the tray to be free-standing.

In some embodiments, the invention is a method of pooling samples located in the wells of a multiwell plate, the method comprising placing the tray having a body with a rectangular top opening, and a closed nozzle, wherein the dimensions of the top opening match the dimensions of a top side of a standard multiwell plate with the wells of the plate facing into the tray, on top of the multiwell plate, and inverting the microwell plate thereby collecting the pooled samples in the tray. The method may further comprise placing the tray and the inverted multiwell plate into an accessory container. The method may further comprise a step of subjecting the tray and the inverted microwell plate to centrifugal force.

In some embodiments, the invention is a method of detecting multiple targets in a plurality of individual cells, the method comprising: binding to the targets in a plurality of cells in a sample a plurality of unique binding agents, each agent specific for one of the targets; splitting the sample into wells of a multiwell plate, each well containing a different subcode oligonucleotide; in the wells, attaching the first subcode oligonucleotide to each of the bound unique agents in the plurality of cells; placing a collection tray having a body with a rectangular top opening, and a closed nozzle, wherein the dimensions of the top opening match the dimensions of a top side of a standard multiwell plate with the wells of the plate facing into the tray, atop the multiwell plate, inverting the tray and the multiwell plate combination and collecting a pooled sample in the bottom of the tray; repeating the steps of splitting, attaching and collecting with the pooled sample from the tray wherein the subcode oligonucleotides in each round of attaching anneal adjacently to the subcode oligonucleotide from a previous round via an annealing region, and are covalently linked to the subcode from the prior round thereby assembling a unique cell-associated barcode on each bound unique agent in each cell. The method may further comprise centrifugation of the tray-inverted microwell plate combination. In some embodiments of the method, the unique agent is an antibody conjugated to a nucleic acid sequence identifying the antibody and the unique cell-associated barcode is attached to the antibody-identifying sequence. In other embodiments of the method, the unique agent is a nucleic acid probe. In some embodiments of the method, multiple different unique agents are used, e.g., a combination of at least one antibody and at least one nucleic acid probe. In some embodiments the method further comprising a step of sequencing the unique cell-associated barcodes.

In some embodiments, the invention is a method of forming a normalized pool of nucleic acid libraries from multiple samples, the method comprising: providing a multiwell plate, wherein each well contains the same amount of a nucleic library consisting of nucleic acid molecules from a sample, each molecule having a sample identification barcode; placing a collection tray described herein atop the multiwell plate, inverting the tray and multiwell plate combination and collecting a normalized pool of nucleic acid libraries in the bottom of the tray.

The method may further comprise withdrawing an aliquot of the normalized pool of nucleic acid libraries and sequencing the libraries. Prior to sequencing, the volume of the normalized pool of nucleic acid libraries may be reduced by a method selected from ethanol precipitation, SPRI bead binding and evaporation, or the volume increased by addition of a diluent.

In some embodiments, the multiwell plate for pooling is prepared by a method comprising: providing a preparatory multiwell plate wherein each well comprises a solution containing a nucleic library consisting of nucleic acid molecules from a sample, each molecule having a sample identification barcode; determining concentration of nucleic acids in the wells of the preparatory multiwell plate;

equalizing the concentration of nucleic acids among wells of the preparatory multiwell plate by adding diluent to the wells as needed; and aliquotting the same volume of the solution from the wells of the preparatory multiwell plate into the corresponding wells of a new multiwell plate. The concentration of nucleic acids may be determined by fluorescence absorbance measurement.

### BRIEF DESCRIPTION OF THE DRAWINGS

The skilled artisan will understand that the drawings included herein are for illustration purposes only. The drawings are not intended to limit the scope of the invention in any way.

FIG. 1 is a perspective view of a cross-section of an assembly with an accessory frame.

FIG. 2 is a perspective view of a cross-section of another embodiment without the accessory frame where the tray has support legs.

FIG. 3 is a perspective view of the assembly placed inside a centrifuge bucket.

FIG. 4 is a perspective view of the free-standing tray.

FIG. 5 is a stacked view of the components of the assembly.

FIG. 6 is a perspective view of a cross-section of the assembly.

FIG. 7 is a front view of an assembly with a tray having a shallow slope of the body allowing for the assembly to be contained within a centrifuge bucket.

### DETAILED DESCRIPTION OF THE INVENTION

The term “quantum barcoding” or “QBC” refers to a method of single-cell analysis called Quantum Barcoding (QBC) (see U.S. Pat. No. 10,144,950) where each cell in a cell mixture is labeled with a unique combinatorial barcode. The barcode is assembled via a split-pool process where a cell suspension is subjected to multiple rounds of mixing, pooling and splitting into multiple containers or wells. The splitting is performed using a microfluidic device or robotic or manual pipetting. Assembling a unique combination barcode required a sufficient number of split-pool rounds so that the number of different possible barcodes matches or exceeds the number of cells.

The terms “multiwell plate” and “microplate” are used interchangeably to refer to a sample device comprising multiple test-tube-like compartments joined together at the top to form a structure, typically rectangular, having multiple well opening arranged in rows and columns, see e.g., U.S. Pat. Nos. 4,734,192 and 5,009,780, 5,141,719.

Multiwell plates are widely used for sample preparation and purification. These plates are also useful for combinatorial synthesis where multiple reactions with multiple compounds occur at once to generate a variety of products.

Multiwell plates contain a plurality of individual wells or reaction chambers; see U.S. Pat. Nos. 4,734,192 and 5,009,780, 5,141,719 for example. The microwell format of the multiwell plate has proven to be convenient for sample processing such as pipetting, washing, shaking, detecting, storing, etc. In some applications, multiwell plates are subjected to incubations at various temperatures including thermocycling protocols. The multiwell plates are also suitable for freezing and storage of samples contained therein. Some instruments such as incubation chambers and thermocyclers have been modified or specially designed to accommodate multiwell plates. In some applications, centrifugation of multiwell plates is required. To that end, there are accommodating centrifuge buckets capable of enclosing or supporting multiwell plates.

A typical protocol involving multiwell plates involves placing the material in the wells of the plate and withdrawing the material from the plate. This is most often performed by a robotic or manually-operated multichannel pipettor. Some lab protocols requiring aspiration of liquids from multiwell plates simply call for inverting the plates to remove small amounts of liquid by the force of gravity. Other protocols require intentional collection and subsequent use of the contents of the multiwell plates.

The instant invention enables pooling and quantitative collection of the contents of multiwell plates. The invention and its applications are not limited to any particular protocol or any type of reactants or cells. For illustration only, the invention is applied to one particular method of single-cell analysis described in U.S. Pat. No. 10,144,950 and termed Quantum Bar Coding or QBC. Briefly, the method relies on a process of split-pool wherein a sample is split into containers (e.g., wells of a multiwell plate), pooled into a single container and split again into a new multiwell plate.

The instant invention is a tray and tray assembly especially useful in the sample-pooling step involving a multiwell plate. Described in general terms, the tray of the instant invention is a device placed atop the multiwell plate with the wells facing into the tray. The plate-tray assembly is then inverted to allow the contents to be pooled in the bottom of the tray nozzle. The bottom of the tray nozzle is a closed, sealed volume. In some embodiments, the tray is supported by an optional accessory frame. In other embodiments, the tray is freestanding by virtue of support legs. The entire assembly comprising the multiwell plate and the tray can be placed into an accommodating centrifuge bucket for collecting the pooled contents in the bottom of the tray by low-speed centrifugation, e.g., 100 s of g. With the use of centrifugation, the contents of the multiwell plate are quantitatively recovered in the bottom of the tray nozzle.

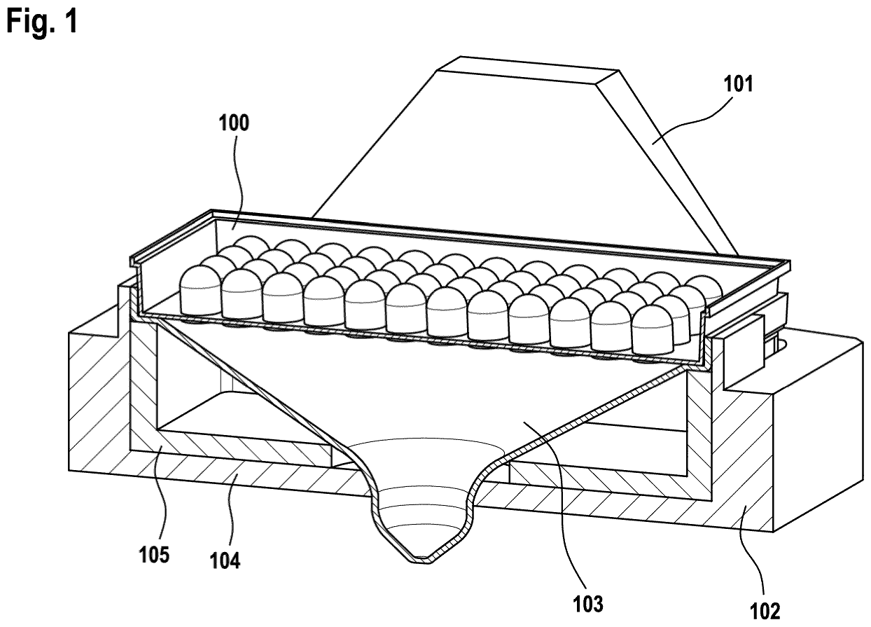

Exemplary embodiments of the invention are now described in more detail in reference to FIGS. 1-6.

FIG. 1 is a perspective view of a cross-section of an assembly comprising centrifuge bucket, an accessory frame, a multiwell plate and a tray. An inverted microwell plate 100 is placed atop a tray 103 supported by accessory frame 105. The combination is placed in a centrifuge bucket 101, having walls 102 and bottom 104.

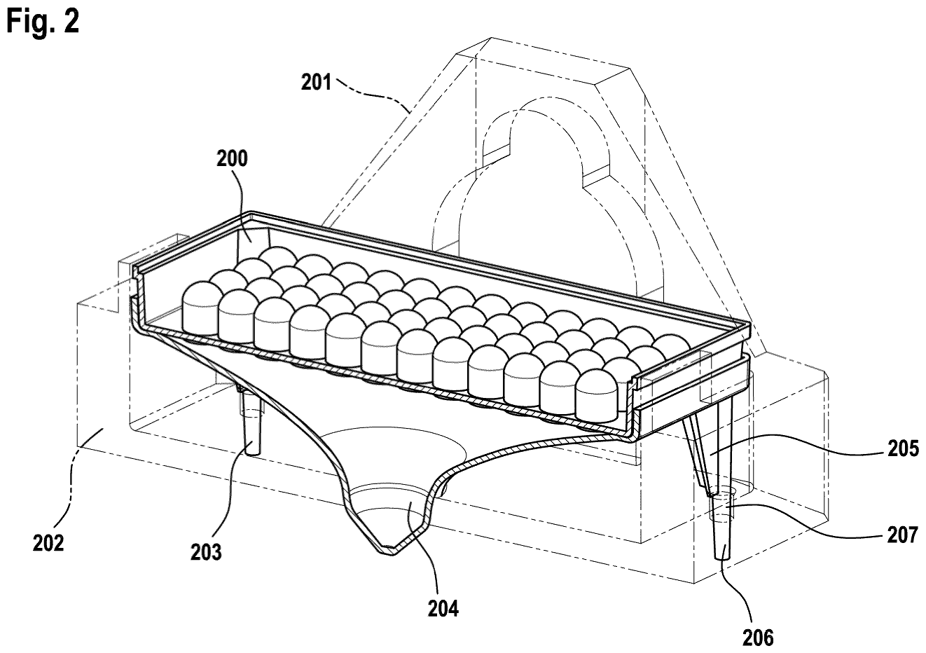

FIG. 2 is a perspective view of a cross-section of another embodiment of the assembly that does not have an accessory frame. The tray is not supported by the accessory frame as in FIG. 1, but instead has supporting legs.

The inverted multiwell plate 200 is placed atop the tray 204 placed in the centrifuge bucket 201. The tray has legs 203, 206 with flanges 205, extended from the legs and from body of the tray. The legs and flanges are illustrated as visible through the side 202 of the centrifuge bucket 201. The bottom of the centrifuge bucket may have a hole 207 to accommodate the legs.

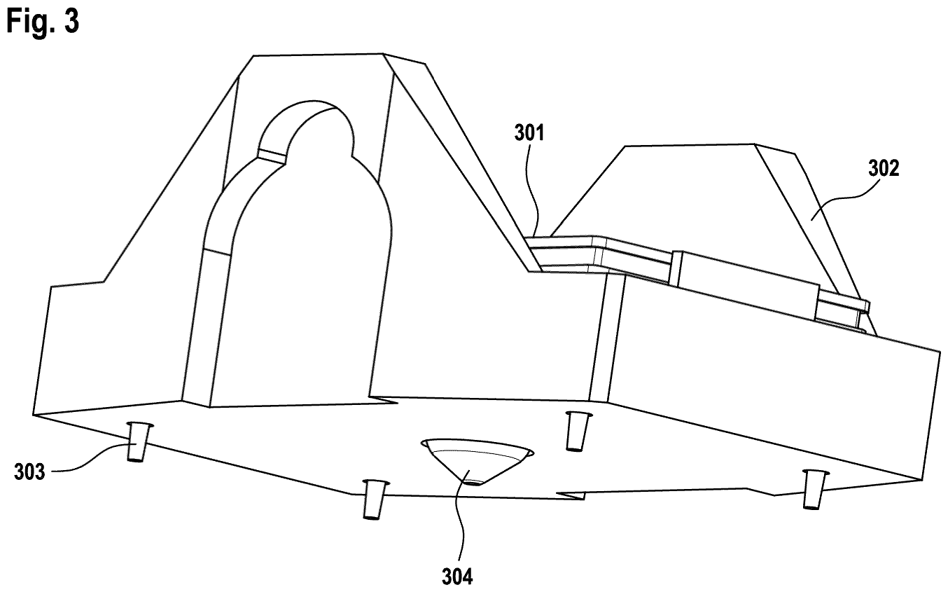

FIG. 3 is a perspective view of the assembly where the following features are visible: multiwell plate 301, centrifuge bucket 302, the support legs of the tray 303, the nozzle 304 of the tray 302. In some embodiments, the accessory frame, the centrifuge bucket or both may have alignment holes for inserting the legs for support and alignment. In some embodiments, the accessory frame, the centrifuge bucket or both may have a hole for inserting the bottom of the nozzle 304 of the tray 302 for additional support and alignment.

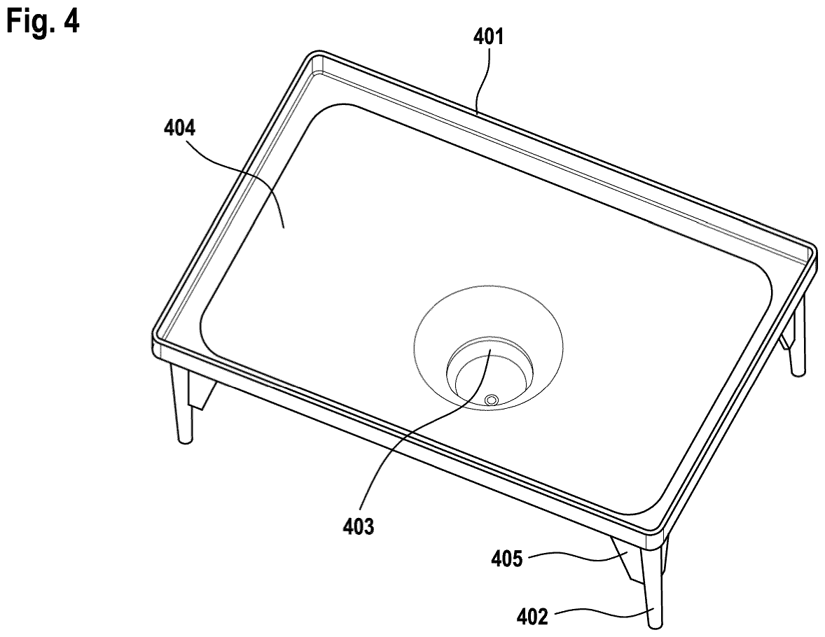

FIG. 4 is a perspective view of the freestanding tray having legs. This embodiment of the tray can be used without an accessory frame. Tray 401 has four legs 402 with flanges 405 extended from the legs and from body of the tray, a closed nozzle 403, and a top opening 404. Each leg has two supporting flanges 405.

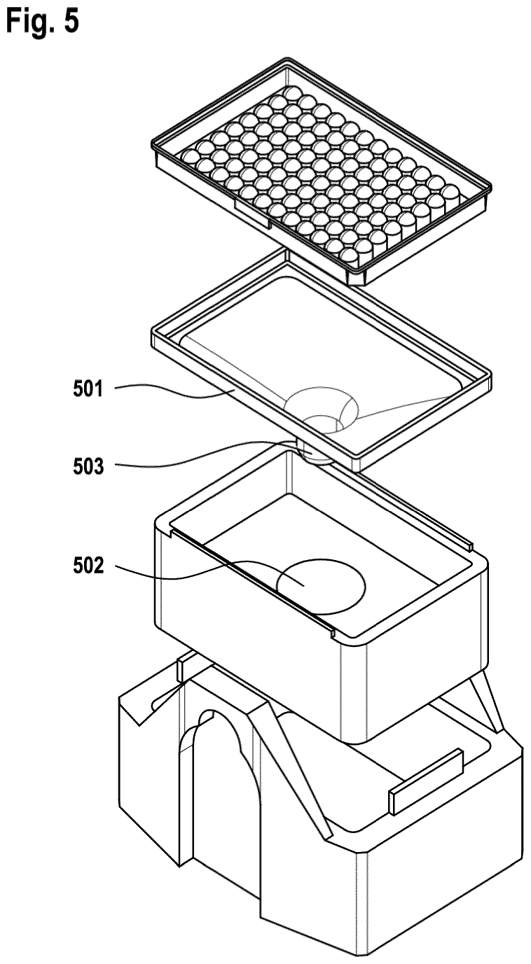

FIG. 5 is a stacked view of the components of the assembly including the multiwell plate, a tray, an optional accessory frame and an exemplary centrifuge bucket. The tray 501 has a nozzle 503 fitting into a space 502 in the accessory frame.

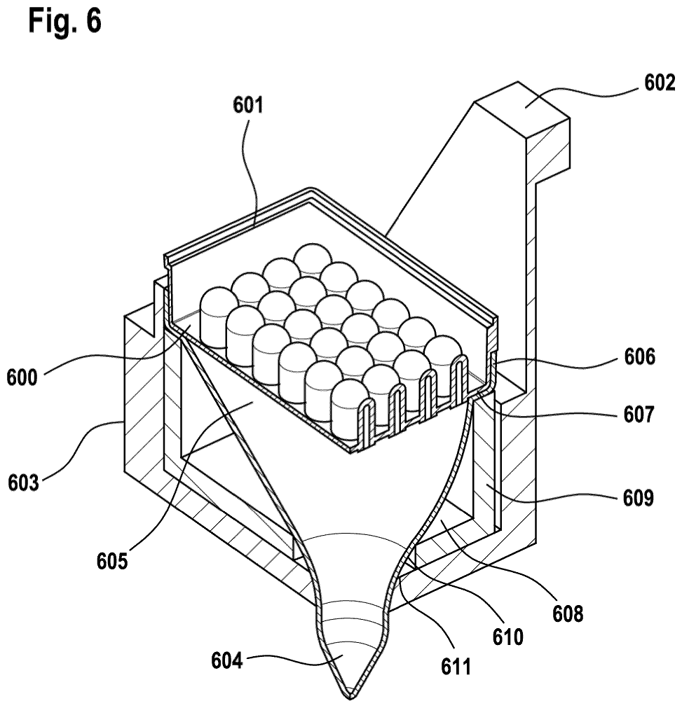

FIG. 6 is a perspective view of a cross-section of one embodiment of the invention. The invention is described in detail below with reference to FIG. 6.

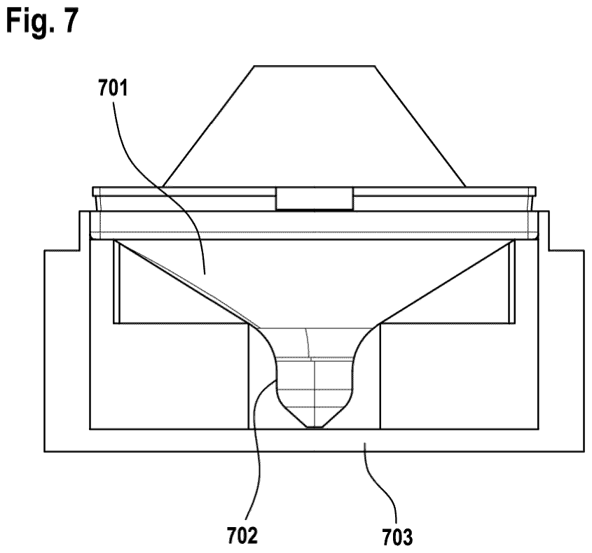

FIG. 7 shows an embodiment of a tray with a shallower slope of the body 701 wherein the nozzle 702 of the tray fits into the centrifuge bucket 703.

Described now in more detail, the invention is a device and an assembly for pooling and quantitatively collecting samples from a multiwell plate. The device and the assembly are also useful for pooling and collecting samples present in multiwell plates by centrifugation.

In reference to FIG. 6, the multiwell plate has a lip 600. The lips is a flat surface in plane with the openings of the wells and extending beyond the area containing the openings of the wells. The multiwell plate further has a shoulder 601. The shoulder is a surface perpendicular to the surface containing the openings of the wells and extending in the same direction as the bodies of the wells. The shoulder has an optional skirt. The skirt is a surface in plane with the shoulder 601 but thicker than the shoulder allowing the plate to rest atop another element placed underneath the skirt.

Referring further to FIG. 6, the multiwell plate rests atop the tray 605. The tray has a nozzle 604. The tray has a lip 607 fitting underneath the lip 600 of the multiwell plate. The lip 600 of the multiwell plate has a width defined as a distance from the outer wells to the shoulder of the multiwell plate. The lip 607 of the tray has a width defined as the distance between the slope of the body of the tray and the shoulder 606 of the tray. The lip 607 of the tray is equal to or greater than the lip 600 of the multiwell plate allowing the plate to rest atop the tray with the wells facing into the body of the tray.

The tray also has a shoulder 606 fitting outside the shoulder 601 of the multiwell plate. The optional skirt of the multiwell rests atop the shoulder 606 of the tray. The dimensions and shape of the tray correspond to the dimensions and shape of the multiwell plate so that the inverted plate can rest atop the tray as shown in FIG. 6. The multiwell plate is considered resting atop the tray if at least a part of the lip 600 of the plate overlaps with the lip 607 of the tray.

Referring further to FIG. 6, in one embodiment, the tray 605 does not have supporting legs as in other embodiments (shown in FIGS. 2, 3 and 4). In the embodiment shown in FIG. 6, the multiwell plate-tray assembly rests atop an accessory frame 608. The frame 608 has the dimensions and shape corresponding to the dimensions and shape of the microwell late and the tray so that the frame can support the plate-tray assembly. The multiwell plate-tray assembly is considered resting atop the accessory frame if at least a part of the accessory frame wall 609 overlaps with the lip 600 of the microwell plate and the lip 607 of the tray.

As shown in FIG. 6, the accessory frame may support only the lip 601 of the multiwell plate resting atop the lip 608 of the tray. In some embodiments, e.g., as shown in FIG. 5, the accessory frame may also partially support the sloped body of the tray 501 and have an opening 502 for the nozzle of the tray 503.

Referring again to FIG. 6, the assembly further may be placed in a centrifuge bucket 602 (having a sidewall 603) shown to enclose the tray, the multiwell plate and the accessory frame. In the embodiment shown in FIG. 6, the accessory frame and the centrifuge bucket are shown to have holes 610 and 611 respectively to accommodate the nozzle of the tray. One of skill in the art will appreciate than the shape of the tray, e.g., the angle of the tray body can be made more shallow to allow for the tray to be inserted into existing centrifuge buckets without the need for a hole 611. It is noted however that for applications requiring quantitative recovery of cells, shallow walls may be disadvantageous as they may cause unwanted adhesion of cells. While a steeper angle of the tray body 606, while resulting in a tray too long for some existing centrifuge buckets, may enable better cell recovery.

FIG. 7 shows an embodiment of a tray with a shallower slope of the body 701 wherein the nozzle 702 of the tray fits into the centrifuge bucket 703.

One of skill in the art will further appreciate that it is advantageous to give the nozzle of the tray 607 dimensions similar to those of existing laboratory containers for which racks and other convenient holder devices exist. In some embodiments, the nozzle of the tray 607 has dimensions close to or identical to those of a standard 50 ml test tube.

As can be seen in the figures, the collection tray includes ornamental aspects to the shape of its curved surfaces. The angles and contours of how the body of the collection tray transitions to the nozzle include functional and ornamental design aspects. The shape that is shown in the figures is one specific form and other geometries are within the contemplation of the inventors herein that would enable similar or same functionality. Other shapes with the same functionality are also contemplated for the support legs, the flanges of the support legs, the lip and the shoulder of the tray. For example, the lip and the shoulder of the tray may provide surfaces for custom labels to be added by the user, or for a manufacturer's trademark or other manufacturer- added information.

Suitable materials for the tray and accessory frame include polymers such as polycarbonates, polyesters, nylons, PTFE resins and other fluoropolymers, acrylic and methacrylic resins and copolymers, polysulphones, polyethersulphones, polyarylsulphones, polystyrenes, polyvinyl chlorides, chlorinated polyvinyl chlorides, ABS and its alloys and blends, polyolefins, preferably polyethylenes or polypropylenes such as linear low density polyethylene or polypropylene, low density polyethylene or polypropylene, high density polyethylene or polypropylene, and ultrahigh molecular weight polyethylene or polypropylene and copolymers thereof, and metallocene generated polyolefins, polyurethanes, and thermoset polymers. Preferred polymers are polyolefins, in particular polyethylenes, polypropylenes and their copolymers, polystyrenes, polycarbonates and acrylic and methacrylic resins and copolymers nitrile copolymers. Additional suitable materials are

The tray is preferably a single, unitary, unassembled piece made of a polymeric moldable material. In some embodiments, the tray is made by injection molding.

The particular material for the tray may be chosen depending on the application of the instant invention. For example, for applications involving the study of cells in solution, low adhesion materials may be required so that the cells easily collect in the nozzle of the tray and no cells adhere to the sides of the tray during collection. In some embodiments, the tray is made from medical grade polypropylene. Alternatively, for collecting only the supernatants and avoiding the cells, high adhesion materials may be required so that any contaminating cells remain on the walls of the tray and a minimum amount reach the nozzle of the tray.

There are fewer considerations for the choice of material for the accessory frame. The accessory frame can be made from any available polymer (including the same polymer chosen for the tray), or from any suitable metal or metal alloy, polycarbonate, Teflon etc.

Multiwell plates of standard sizes are widely available from multiple manufacturers including Greiner, Eppendorf, ThermoFisher Scientific, Sigma Aldrich, Millipore, Qiagen and many others. The number of wells in commercially available microwell plates ranges from 6 to 1536, however the overall dimensions are similar. A typical microwell plate has a width of 86 mm (3.4 in) and length of 128 mm (about 5 in). Adherence to this industry standard allows the use of the instant invention with any commercially available microwell plate. The particular source and type of a multiwell plate may be chosen depending on the application of the instant invention. For example, for applications involving the study of cells in solution, low adhesion materials may be required. Alternatively, for collecting only the supernatants and not the cells, high adhesion materials promoting cell adhesion and biofilm formation may be used. For example, a flat bottom well such as the Nunc brand (Sigma Aldrich) may be more appropriate.

In some embodiments, the invention is a method of utilizing the novel device disclosed herein to pool and collect samples from multiwell plates. In an exemplary embodiment, each of the wells of a multiwell plate contains a sample or an aliquot of a sample. In some embodiments, a chemical reaction or a bio-chemical reaction or another biological process (e.g., cell growth) takes place. At the completion of there action or event, the samples need to be pooled into a single volume and collected. The invention comprises a step of placing a novel tray described herein on top of the multiwell plate with the wells facing into the body of the tray. Next, the multiwell plate-tray combination is inverted. In some embodiments, the multiwell plate tray combination is free-standing, i.e., can stand on its own resting on the legs of the tray (FIG. 4). In other embodiments, the multiwell plate-tray combination is placed into an accessory frame (FIG. 1).

The multiwell plate may be left to rest atop the tray until the pooled sample is collected in the bottom of the tray by gravity. In other embodiments, the multiwell plate-tray combination is placed into a centrifuge bucket adapted to house multiwell plates. The pooled sample is collected in the nozzle of the tray and is withdrawn, e.g., by pipetting for further processing.

In some embodiments, the cells or extracellular milieu is particularly conducive to adhesion of cells to solid surfaces. In such instances, the body of the tray has a steep angle desired for maximum collection of the sample and minimal adhesion of the sample to the walls of the tray. In such embodiments, the centrifuge bucket needs an aperture to accommodate the nozzle of the tray. (as shown in FIG. 1).

In other embodiments, the materials to be handled, i.e. solutions of small molecules or uniformly charged molecules such as nucleic acids are not prone to adhesion. In such instances, the body of the tray may have a shallower angle so that the entire tray including the nozzle fits into a standard centrifuge bucket with no modifications to the bucket. (as shown in FIG. 7)

As an example, the novel device disclosed herein is used to perform quantum barcoding (QBC) described in U.S. Pat. 10,144,950, which is incorporated herein by reference.

The application described below involves the use of a sample. In some embodiments, the sample is derived from a subject or a patient. In some embodiments the sample may comprise a fragment of a solid tissue or a solid tumor derived from the subject or the patient, e.g., by biopsy. The sample may also comprise body fluids (e.g., urine, sputum, serum, plasma or lymph, saliva, sputum, sweat, tear, cerebrospinal fluid, amniotic fluid, synovial fluid, pericardial fluid, peritoneal fluid, pleural fluid, cystic fluid, bile, gastric fluid, intestinal fluid, or fecal samples) that may contain cells. The sample may comprise whole blood or blood fractions where normal or tumor cells may be present. In other embodiments, the sample is a cultured sample, e.g., a tissue culture containing cells. In some embodiments, the cells of interest in the sample are infectious agents such as bacteria, protozoa or fungi.

In other embodiments, the sample is a chemical reaction mixture comprising a first reactant to be introduced to an array of second reactants, each of the second reactants isolated in a well of a multi-well plate.

In the context of the exemplary application described below, nucleic acids, proteins or other markers of interest may be present in the cells and are the target of the cell-handling procedure. Each nucleic acid target is characterized by its nucleic acid sequence. Each protein target is characterized by its amino acid sequence and its epitopes recognized by specific antibodies. In some embodiments, the target nucleic acid contains a locus of a genetic variant, e.g., a polymorphism, including a single nucleotide polymorphism or variant (SNP of SNV), or a genetic rearrangement resulting e.g., in a gene fusion. In some embodiments, a protein biomarker contains an amino-acid change resulting in the creation of a unique epitope. In some embodiments, the target nucleic acid or target protein comprises a biomarker, i.e., a gene or protein antigen whose variants are associated with a disease or condition. For example, the target nucleic acids and proteins can be selected from panels of disease-relevant markers described in U.S. patent application Ser. No. 14/774,518 filed on Sep. 10, 2015. Such panels are available as AVENIO ctDNA Analysis kits (Roche Sequencing Solutions, Pleasanton, Calif.) In other embodiments, the target nucleic acids or proteins are characteristic of a particular organism and aids in identification of the organism or a characteristic of the pathogenic organism such as drug sensitivity or drug resistance. In yet other embodiments, the target nucleic acid or protein is a unique characteristic of a human subject, e.g., a combination of HLA or KIR sequences defining the subject's unique HLA or KIR genotype. In yet other embodiments, the target nucleic acid is a somatic sequence such as a rearranged immune sequence representing an immunoglobulin (including IgG, IgM and IgA immunoglobulin) or a T-cell receptor sequence (TCR).

In yet another application, the target is a fetal sequence present in maternal blood, including a fetal sequence characteristic of a fetal disease or condition or a maternal condition related to pregnancy. For example, the target could be one or more of the autosomal or X-linked disorders described in Zhang et al. (2019) Non-invasive prenatal sequencing for multiple Mendelian monogenic disorders using circulating cell-free fetal DNA, Nature Med. 25(3):439.

In some embodiments, the target is a nucleic acid (including mRNA, microRNA, viral RNA, cellular DNA or cell-free DNA (cfDNA) including circulating tumor DNA (ctDNA)).

In some embodiments, the target is a protein expressed in the cell. For example, the protein target may be cell-surface protein. In some embodiments, the cell surface protein is a lymphocyte surface protein selected from inhibitory receptors (such as Pdcd1, Havrcr2, Lag3, CD244, Entpd1, CD38, CD101, Tigit, CTLA4), cell surface receptors (such as TNFRSF9, TNFRSF4, Klrg1, CD28, Icos, IL2Rb, IL7R) or chemokine receptors (such as CX3CR1, CCL5, CCL4, CCL3, CSF1, CXCR5, CCR7, XCL1 and CXCL10). In some embodiments, the proteins are selected from CD4, CD8, CD11, CD16, CD19, CD20, CD45, CD56 and CD279.

Briefly, in a typical Quantum Bar Coding (QBC) protocol, cells are isolated from a sample. The cells in a reaction solution are contacted with a unique binding agent, e.g., DNA or RNA probe or an antibody, each unique binding agent being specific for a target in the cell.

The unique binding agent comprises at least one part or element specifically interacting with a target and an element allowing for assembly of a combinatorial barcode. A nucleic acid probe may have target recognition portion and a portion complementary to a portion of the barcode to be attached. An antibody may comprise an immunoglobulin protein with a linker oligonucleotide that facilitates assembly of a barcode, i.e., complementary to a portion of the barcode to be attached. Methods to attach nucleic acids to proteins and specifically to antibodies are known, e.g., Gullberg et al., PNAS 101 (22): pages 228420-8424 (2004); Boozer et al, Analytical Chemistry, 76(23): pages 6967-6972 (2004) or Kozlov et al., Biopolymers 5: 73 (5): pages 621-630 (2004).

In some embodiments, the assay is a multiplex assay whereby a plurality of target molecules is detected in a plurality of cells. For example, a single QBC reaction mixture may contain a plurality of different nucleic acid probes, or a plurality of different antibodies, or a combination of nucleic acid probes and antibodies. The unique binding agent may be an aptamer, including a nucleic acid aptamer (i.e., single-stranded DNA molecules or single-stranded RNA molecules) and a peptide aptamer.

Next, the cells with a bound unique binding agent (e.g., an antibody or a nucleic acid probe) is subjected to a split-pool process to assemble a unique barcode on each cell. Each unique cell-originating code is assembled on each cell where a unique binding agent has bound. The unique barcode is a modular structure assembled from subunits by the process of stepwise addition. The subunits attach to each other or to a common backbone via attachment regions, e.g., complementary nucleic acid sequences. The attachment may comprise one or both of hybridization and ligation to the backbone or to the adjacent code subunit.

The assembly of unique barcodes is accomplished via split-pool process. Each round of split pool synthesis comprises i) splitting the population of cells into wells of a multiwell plate wherein each well comprises a barcode subunit (subcode); ii) attaching the subcode to the nascent code in each of the wells; and iii) pooling the reaction volumes; and repeating steps i-iii with fresh multiwell plates where each well contains a new subcode. The first subcode is attached to the nucleic acid probe, or a nucleic acid linker attached to the antibody or the nucleic acid probe. Non-nucleic acid linkers can also be used to attach the first subcode. Subsequent subcodes re attached to subcodes from the previous round of code assembly. Through multiple rounds of split-pool process, each cell follows a unique path through the wells of a series multiwell plates containing different subcodes. After a sufficient number of split pool rounds, enough subunits are added to generate enough diversity to ensure a statistical likelihood that no two cells have the same barcode. As a result, each cell acquires a unique combinatorial barcode composed of subcodes arranged in a unique combination. A more detailed description of the QBC method and its applications can be found in Nolan, G., et al., (2020) “Ultra-high throughput single-cell analysis of proteins and RNAs by split-pool synthesis,” Communications Biology, In Press.

In the context of the instant invention, step iii of the QBC process comprises placing the tray described herein atop the multiwell plate containing the cells combined in each well with a subcode. Next, the multiwell plate-tray combination is inverted and placed into a bucket of a centrifuge accommodating multiwell plates. Optionally, the inverted multiwell plate-tray combination is first placed into an accessory frame and then into a bucket of a centrifuge. After centrifugation at low speed (e.g., 100 g, 200 g, 300 g, 400 g or 500 g), the pooled sample is collected into the nozzle of the tray. The pooled samples are withdrawn from the nozzle of the tray and dispensed into the wells of the next multiwell plate and the process is repeated. Optionally, the pooled sample is dispensed into the wells of the next multiwell plate by a multichannel pipettor. At the completion of the barcode assembly, the last sample collected in the tray nozzle is subjected to nucleic acid extraction and sequencing of the nucleic acid barcodes from each cells.

The unique cellular barcodes assembled as described herein can be subjected to nucleic acid sequencing. Sequencing can be performed by any method known in the art. Especially advantageous is the high-throughput single molecule sequencing method utilizing nanopores, including by a method involving threading through a biological nanopore (U.S. Pat. No. 10,337,060) or a solid-state nanopore (U.S. Pat. No. 10,288,599, US20180038001, U.S. Pat. No. 10,364,507), or by a method involving threading tags through a nanopore (U.S. Pat. No. 8,461,854) or any other presently existing or future DNA sequencing technology utilizing nanopores.

Other suitable technologies of high-throughput single molecule sequencing. include the Illumina HiSeq platform (Illumina, San Diego, Calif.), Ion Torrent platform (Life Technologies, Grand Island, N.Y.), Pacific BioSciences platform utilizing the Single Molecule Real-Time (SMRT) technology (Pacific Biosciences, Menlo Park, Calif.) or a platform utilizing nanopore technology such as those manufactured by Oxford Nanopore Technologies (Oxford, UK) or Roche Sequencing Solutions (Santa Clara, Calif.) and any other presently existing or future DNA sequencing technology that does or does not involve sequencing by synthesis.

The sequencing step may utilize platform-specific sequencing primers. Binding sites for these primers may be added to barcode sequences by amplifying the barcode sequences with primers having a 3′-portion annealing to a common sequence in the last barcode subunit and 5′-portions comprising platform-specific sequences. The final subcode may already comprise a platform-specific sequence needed to introduce the barcodes into the sequencing platform.

As another example, the novel device disclosed herein is used to prepare a normalized pool of nucleic acid libraries for sequencing. A typical sequencing workflow involving massively parallel sequencing of individual molecules includes a step of pooling samples. A sample is represented by a library of nucleic acid molecules from the sample, where each molecule has a sample identification barcode (SID). A user must combine (pool) multiple libraries, into a single pool that gets loaded onto a sequencer flow cell. The goal is to pool equal parts (or well- defined ratios) of sample libraries so that each sample is read at a desired depth, i.e., the needed number of sequence reads. Equal read depth is essential for getting credible sequence information from a sample.

To date, existing solutions involve sampling libraries to be pooled into multiwell plates (e.g., 24, 48, 96, 384, or 768 well plates) and measuring the concentration of nucleic acid in each well by fluorescence absorption. Convenient devices exist for measuring absorption directly in translucent multiwell plates (e.g., from Molecular Devices, San Jose, Calif.) After the concentration of nucleic acids has been determined, the concentration is equalized among wells by adding varying volumes of diluent to the wells as needed. The wells of the resulting multiwell plate contain the same concentration of nucleic acids but different volumes of nucleic acid solutions.

The existing solution to forming a normalized pool involves pipetting the same amount of liquid from each well into a common container thus forming a normalized pool of nucleic acid libraries. This can be done manually of with the help of a robot.

In some embodiments, the instant invention is a greatly simplified method of forming a normalized pool of nucleic acid libraries from multiple samples for nucleic acid sequencing. In some embodiments, the method starts by forming a preparatory multiwell wherein each well has a different concentration of nucleic acids from a sample. Nucleic acids are present in the form of a library consisting of a plurality of nucleic acid molecules from a sample, each molecule having a sample identification barcode. Next, the method comprises determining concentration of nucleic acids in the wells of the preparatory multiwell plate. The concentration of nucleic acids may be determined by fluorescence absorbance measurement. The concentration is then equalized among wells of the preparatory multiwell plate by adding diluent to the wells as needed.

Next, the method includes a step of aliquotting the same volume of the solution from the wells of the preparatory multiwell plate into the corresponding wells of a new multiwell plate. The aliquotting may be done by a multi-channel pipettor or a robotic pipettor, including a robotic multichannel pipettor.

Next, the method comprises a step of pooling the libraries using the novel tray and tray assembly described herein. The tray is placed atop the multiwell plate. Next, the method involves inverting the tray and multiwell plate combination and collecting a normalized pool of nucleic acid libraries in the bottom of the tray. The normalized pool or an aliquot of the normalized pool of nucleic acid libraries can be used for sequencing, i.e., loaded onto the flowcell of the sequencer. Prior to sequencing, the volume of the normalized pool of nucleic acid libraries may be reduced by a method selected from ethanol precipitation, SPRI bead binding and evaporation, or the volume increased by addition of a diluent.

The exemplary uses of the novel device and novel assembly set forth above are not limiting. Rather, the novel device and novel assembly facilitating the split pool process find applications in any process where combinatorial synthesis is desired. The device and assembly of the invention can be used in any diagnostic, prognostic, therapeutic, patient stratification, drug development, treatment selection, and screening process that involves a split-pool process including splitting a sample into the wells of a multi-well plate and pooling the split samples back into a single reaction vessel or reaction volume.

While the invention has been described in detail with reference to specific embodiments and examples, it will be apparent to one skilled in the art that various modifications can be made within the scope of this invention. Thus the scope of the invention should not be limited by the examples described herein, but by the claims presented below.

1. A sample collection device comprising: a standard multiwell base plate, and a tray placed on the side of the multiwell plate containing the well openings, the multiwell plate having a lip, a shoulder and a skirt, the tray having a lip, a shoulder, a body and a closed nozzle, wherein, the lip of the plate rests atop the lip of the tray and the shoulder of the plate is enclosed by the shoulder of the tray and the shoulder of the tray abuts the skirt of the plate.

2. The device of claim 1, wherein the lip of the plate and the lip of the tray each have a width and the width of the tray lip is not less than the width of the plate lip.

3. The device of claim 1, wherein the tray further comprises legs extending from the body in the direction of the tray nozzle to enable the tray to be freestanding.

4. The device of claim 1, further comprising an accessory frame, having the bottom and four side walls, wherein the lip of the tray rests atop the edge of the side wall and the thickness of the side wall is no less than the width of the tray lip.

5. The device of claim 1, wherein the tray is made from low adhesion polymer material.

6. The device of claim 1, wherein the tray is made from polymer material selected from polycarbonates, polyesters, nylons, fluoropolymers, acrylic and methacrylic resins and copolymers, polysulphones, polyethersulphones, polyarylsulphones, polystyrenes, polyvinyl chlorides, chlorinated polyvinyl chlorides, polyolefins and copolymers thereof and polyurethanes.

7. The device of claim 1, wherein the accessory frame is made from material selected from moldable polymer material, Teflon, polycarbonate or metal.

8. A sample collection tray having a body with a rectangular top opening, and a closed nozzle, wherein the dimensions of the top opening match the dimensions of a top side of a standard multiwell plate with the wells of the plate facing into the tray.

9. The sample collection tray of claim 8, further comprising legs extending from the body in the direction of the nozzle and enabling the tray to be free-standing.

10. A method of pooling samples located in the wells of a multiwell plate, the method comprising placing the tray of claim 8-9 on top of the multiwell plate, and inverting the microwell plate thereby collecting the pooled samples in the tray.

11. A method of detecting multiple targets in a plurality of individual cells, the method comprising:
a) binding to the targets in a plurality of cells in a sample a plurality of unique binding agents, each agent specific for one of the targets;
b) splitting the sample into wells of a multiwell plate, each well containing a different subcode oligonucleotide;
c) in the wells, attaching the first subcode oligonucleotide to each of the bound unique agents in the plurality of cells;
d) placing a collection tray of claim 8-9 atop the multiwell plate, inverting the tray and the multiwell plate combination and collecting a pooled sample in the bottom of the tray;
e) repeating steps b., c., and d. with the pooled sample from step d. wherein the next subcode oligonucleotide in each round of step c. anneals adjacently to the subcode oligonucleotide from a previous round via an annealing region, and are covalently linked to the subcode from the prior round of step c. thereby assembling a unique cell-associated barcode on each bound unique agent in each cell.

12. The method of claim 11, wherein step d. further comprises centrifugation.

13. The method of claim 11, wherein multiple different unique agents are used.

14. A method of forming a normalized pool of nucleic acid libraries from multiple samples, the method comprising:
a) providing a multiwell plate, wherein each well contains the same amount of a nucleic library consisting of nucleic acid molecules from a sample, each molecule having a sample identification barcode;
b) placing a collection tray of claim 8-9 atop the multiwell plate, inverting the tray and multiwell plate combination and collecting a normalized pool of nucleic acid libraries in the bottom of the tray.

15. The method of claim 14, wherein the multiwell plate of step a. is prepared by a method comprising:
a) providing a preparatory multiwell plate wherein each well comprises a solution containing a nucleic library consisting of nucleic acid molecules from a sample, each molecule having a sample identification barcode;
b) determining concentration of nucleic acids in the wells of the preparatory multiwell plate;
c) equalizing the concentration of nucleic acids among wells of the preparatory multiwell plate by adding diluent to the wells as needed; and
d) aliquotting the same volume of the solution from the wells of the preparatory multiwell plate into the corresponding wells of a new multiwell plate.
